(19) United States
(12) Patent Application Publication
(10) Pub. No.: US 2015/0050250 A1
(43) Pub. Date: Feb. 19, 2015

(54) CELL THERAPY TECHNOLOGY TO DELIVER RADIO-PROTECTIVE PEPTIDES

(71) Applicant: UNIVERSITY OF SOUTHERN CALIFORNIA, Los Angeles, CA (US)

(72) Inventors: Minnie McMillan, Bradbury, CA (US); Lynn E. Foster, West Hollywood, CA (US)

(21) Appl. No.: 14/390,930

(22) PCT Filed: Apr. 5, 2013
(86) PCT No.: PCT/US2013/035455

§ 371 Date: Oct. 6, 2014

Related U.S. Application Data
(60) Provisional application No. 61/620,904, filed on Apr. 5, 2012.

(51) Int. Cl. C07K 14/81

(52) U.S. Cl. CPC C07K 14/811
USPC 424/93.7; 435/325

(57) ABSTRACT

A method of reducing a symptom of radiation exposure in a subject is provided. The method includes a step of introducing mammalian cells into the subject, the mammalian cells having been treated ex vivo to insert therein a polynucleotide encoding polypeptide that is protective against radiation. The mammalian cells express in vivo in the subject a therapeutically effective amount of the polypeptide thereby reducing a symptom of radiation exposure.

### CROSS-REFERENCE TO RELATED APPLICATION

This application claims the benefit of U.S. provisional application Ser. No. 61/620,904 filed Apr. 5, 2012, the disclosure of which is incorporated in its entirety by reference herein.

### SEQUENCE LISTING

The text file Sequence Listing 0115-sequence_ST25.txt, created Apr. 4, 2013, and of size 2 KB, filed herewith, is hereby incorporated by reference.

### FIELD OF THE INVENTION

The present invention relates to radio-protective cell therapeutic methods.

### BACKGROUND OF THE INVENTION

An urgent need exists for drugs that protect against exposure to radiation in the context of terrorism, nuclear accident or radiological or nuclear attacks during warfare. Currently, emergency response personnel or “first responders” have no protection from the harmful consequences of ionizing radiation exposure. Successful completion of this research could provide first responders and members of the Armed Forces with transient immunity from radiation exposure.

Exposure to ionizing radiation can result in lethality due to hematopoetic damage, gastrointestinal damage, and central nervous system damage. Radiation may cause damage by directly hitting critical targets in the cell such as DNA. Damage may also be indirect—radiation hitting oxygen and water molecules in cells results in production of radical oxygen species (ROS) such as superoxide and hydroxyl radicals that can break chemical bonds and damage DNA; such damage causes cellular differentiation in fibroblasts and apoptotic cell death in endothelial cells resulting in loss or alteration of tissue function. Both dying and surviving cells within an irradiated tissue cell release inflammatory cytokines setting in motion a cascade effect wherein inflammatory cells including lymphocytes, macrophages and polymorphonuclear leukocytes infiltrate tissues causing more cell killing through additional inflammatory cytokines and byproducts, including more ROS.

Compounds that can reduce the deleterious effects of radiation are of interest in the case of accidental or terrorism-related exposures and in the case of protecting normal tissue during therapeutic use of radiation for cancers. Many agents being investigated as potential radioprotectors are antioxidants that scavenge free radicals, thus preventing indirect DNA damage, the predominant cause of cell death after exposure to ionizing radiation. These include amifostine and other thiols, nitroxides, superoxide dismutase mimetics, and melatonin and its homologues. To be effective, these compounds must be present at the time of irradiation. Increased knowledge of the molecular mechanisms of ionizing irradiation-induced cell killing at the level of single cells, tissues and organs has broadened the types of radioprotective agents to include such possibilities as a Toll-like receptor agonist, cytokines and growth factors.

Despite the effort that has gone into the search for radiation protectors or mitigators, only amifostine is in clinical use to prevent xerostomia induced by irradiation, and only potassium iodide is recognized as a radioprotectant in the context of accidental radiation exposure, protecting solely the thyroid from radioactive iodine.

Accordingly, there is a need for improved methods that protect individuals from radiation exposure.

### SUMMARY OF THE INVENTION

Against this prior art background, a radio-protective cell therapeutic method of reducing a symptom of radiation exposure in a subject is provided. The method includes a step of introducing mammalian cells into the subject, the mammalian cells having been treated ex vivo to insert therein a polynucleotide encoding polypeptide that is protective against radiation. The mammalian cells express in vivo in the subject a therapeutically effective amount of the polypeptide thereby reducing a symptom of radiation exposure. Advantageously, the in vivo production of radio-protective peptides to ameliorate radiation damage in first responders in hazardous situations, (whether military or civilian), cannot be overestimated. Similar protection may be equally useful in preserving normal tissue in cancer patients during tumor radiation and even, in the future, the shielding of astronauts from solar radiation. The continuous delivery of the therapeutic agent negates the need for high dose injections which have a greater chance of causing serious adverse side effects.

In another embodiment, a method of reducing a symptom of radiation exposure in a subject is provided. The method includes a step of introducing mammalian cells into the subject. The mammalian cells have been transduced with an expression vector including a polynucleotide encoding polypeptide that is protective against radiation and an expression control sequence operably linked to the polypeptide. The mammalian cells express the polypeptide at least 10% of the polypeptide's amino acid residues polypeptide selected for the group consisting of cysteine, tryptophan, phenylalanine, tyrosine and combinations thereof. Typically, a therapeutically effective amount of the polypeptide is expressed in the subject thereby reducing a symptom of radiation exposure.

In still another embodiment, a device for delivering radiation protecting polypeptides to a subject using the mammalian cells set forth above is provided. The device includes a chamber with mammalian cells sequestered therein. The mammalian cells have been transduced with an expression vector including a polynucleotide encoding polypeptide that is protective against radiation and an expression control sequence operably linked to the polypeptide. The mammalian cells express the polypeptide at least 10% of the polypeptide's amino acid residues polypeptide selected for the group consisting of cysteine, tryptophan, phenylalanine, tyrosine and combinations thereof. Typically, a therapeutically effective amount of the polypeptide is expressed in the subject implanted with the device thereby reducing a symptom of radiation exposure.

In yet another embodiment, a cultured cell useful in the methods and devices set forth above is provided. The cultured cell includes a polynucleotide encoding polypeptide that is protective against radiation. At least 10% of the polypeptide's amino acid residues are selected for the group consisting of cysteine, tryptophan, phenylalanine, tyrosine and combinations thereof. An expression control sequence is operably linked to the polynucleotide such that the cultured cell expresses the polypeptide.

### BRIEF DESCRIPTION OF THE DRAWINGS

Exemplary embodiments of the present invention will become more fully understood from the detailed description and the accompanying drawings, wherein:

FIG. 1 provides a schematic illustration of a TheraCyte™ implantable device;

FIG. 2 provides a schematic of radioprotection protocol;

FIG. 3 provides a plot of survival of transduced human fibroblasts contained within a TheraCyte device and implanted in mice; and

FIG. 4 provides diagrams of retroviral vectors.

### DESCRIPTION OF THE INVENTION

Reference will now be made in detail to presently preferred compositions, embodiments and methods of the present invention which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

The term “subject” as used herein refers to a human or animal, including all mammals such as primates (particularly higher primates), sheep, dog, rodents (e.g., mouse or rat), guinea pig, goat, pig, cat, rabbit, and cow. A subject is sometimes referred to herein as a “patient.”

The term “operably linked” are used in at least one embodiment, means a functional linkage between the expression control sequence and the coding sequence to which it is linked. The operable linkage permits the expression control sequence to control expression of the coding sequence. Expression control sequences can include a promoter, a transcriptional activator binding sequence, an enhancer sequence or any other regulatory or non-regulatory sequence that may be required for transcription and translation of the coding sequence to which the expression control sequence is linked.

The term “radio-protective peptide” are used in at least one embodiment refers to a polypeptide that ameliorate radiation damage in a subject.

The term “gene” are used in at least one embodiment refers to a deoxyribonucleotide sequence coding for an amino acid sequence.

The term “mini-gene” are used in at least one embodiment refers to a deoxyribonucleotide sequence coding for a mini-protein.

The term “mini-protein” are used in at least one embodiment refers to an expressed polypeptide sharing homology, regardless of size or region, with a full protein.

In at least one aspect, the present invention provides a radio-therapeutic method. The method includes a step in which a gene designed to encode and secrete at least one radio-protective peptide is transduced into mammalian cells. In a refinement, the cells are sequestered in a chamber, in order to protect them from the immune response of a recipient and to allow straightforward removal at the end of a deployment. Examples of mammalian cells that may be used include, but are not limited to, fibroblasts, autologous B cells, stem cells, and the like. The mammalian cells are introduced into the subject where they express the polypeptide. In at least one refinement, a therapeutically effective amount of the polypeptide is expressed thereby reducing a symptom of radiation exposure. The method of the present embodiment is not confined to one peptide sequence but allows for the use of multiple peptides thereby offering the broadest means of combating the damage caused by radiation.

In a refinement of the present embodiment, the polypeptide that is protective against radiation includes at least 2 amino acid residues selected from the group consisting of cysteine, tryptophan, phenylalanine, tyrosine and combinations thereof. In another refinement, the polypeptide that is protective against radiation includes at least 3 amino acid residues selected from the group consisting of cysteine, tryptophan, phenylalanine, tyrosine and combinations thereof. In still another refinement, the polypeptide that is protective against radiation includes at least 3 amino acid residues polypeptide selected for the group consisting of cysteine, tryptophan, phenylalanine, tyrosine and combinations thereof. Typically, the polypeptide that is protective against radiation includes from 2 to 12 amino acid residues selected from the group consisting of cysteine, tryptophan, phenylalanine, tyrosine and combinations thereof. Generally, the polypeptide that is protective against radiation includes at least 10% of the amino acid residues selected from the group consisting of cysteine, tryptophan, phenylalanine, tyrosine and combinations thereof. It should also be appreciated that the polypeptide may encode a complete protein such as the Bowman Birk protease inhibitor (BBI) or a fragment thereof. Bowman Birk protease inhibitor (BBI) has the following amino acid sequence:

SEQ ID NO: 1
DDESSKPCCD QCACTKSNPP QCRCSDMRLN SCHSACKSCI
CALSYPAQCF CVDITDFCYE PCKPSEDDKE N

Protein fragments that are useful in the present embodiment may include from about 10 to 40 amino acid residues. A particularly useful protein fragment of the Bowman Birk protease inhibitor has the following sequence:

SEQ ID NO: 2
CALSYPAQCFC

The method of the present invention is advantageously used to provide soldiers and/or first responders with protection from radiation exposure. In such instances, a subject receives an implant encapsulating cells secreting a radio-protective peptide that would give them some protection from radiation exposure. In a refinement, the device is administered subcutaneously before possible exposure to ionizing radiation. In a further refinement, the device continuously delivers protection for at least three months before being removed. Our strategy is not confined to one peptide sequence but allows for the use of multiple peptides thereby offering the broadest means of combating the damage caused by radiation. The sequestration of transduced fibroblasts within an implantable device ensures that the cells are not destroyed by the immune responses of subjects (i.e., patients), that the same “universal” cell line can be used for every recipient and that, in the event of adverse effects, the devices can be rapidly removed.

In a refinement, the radio-protective peptides include peptides that are cysteine containing peptides that can scavenge free radicals. In another refinement, the radio-protective peptides include multiple cysteines, multiple tryptophans, multiple phenylalanine, multiple tyrosines, and combinations thereof. Genes encoding these peptides are designed for secretion, synthesized, cloned and used to transduce fibroblasts. In another refinement, signal sequences targeting other cellular locations (e.g., the nucleus and the mitochondria) are utilized. In still another refinement, minigenes encode ‘area-code’ motifs (i.e., signal sequences). Such signal sequences include sequences that target caveolae, endocytosis, and membranes (including the TAT sequence derived from HIV) are utilized. For example, the Bowman Birk protease inhibitor (BBI) is a soybean-derived polypeptide of 71 residues that protects normal cells against ionizing and ultraviolet radiation through effect on the repair of irradiation induced DNA damage by nucleotide excision repair (NER) and repair of double-stranded breaks (DSB). BBI is a particularly attractive candidate for drug development since it has been shown to provide selective radioprotection to normal tissue in vivo and could, therefore, also be clinically relevant.

In accordance with the methods set forth above, a DNA expression vector which expresses the radio-protective polypeptide in a host (e.g. human for human subjects) mammalian cell is constructed. The expression vector is then introduced into the mammalian cell that subsequently expresses the radio-protective polypeptide therein. The expression vector is inserted into the mammalian cell using a gene transfer procedure. Examples of such procedures include, but are not limited to, RNA viral mediated gene transfer (e.g., retroviral transduction), DNA viral mediated gene transfer, electroporation, calcium phosphate mediated transfection, liposome mediated gene transfer, or microinjection.

As set forth above, some embodiments of the invention have expression vectors that include a nucleic acid encoding the radio-protective polypeptides described herein. The term “expression vector” refers to a nucleic acid molecule capable of transporting another nucleic acid to which it has been linked. In one refinement, the vector is capable of autonomous replication. In another variation, the vector integrates into a host DNA. Those skilled in the art of molecular biology will readily recognize that a number of expression vectors are successfully used in the present embodiment. An expression vector contains functional components required for the production of polypeptides of interest. This includes a suitable RNA polymerase promoter to direct transcription of the gene of interest; transcription termination sequences after the gene of interest to terminate transcription; and translation initiation sequences prior to the gene of interest to promote translation of the gene of interest. Examples of useful expression vectors include, but are not limited to, plasmid vectors and viral vectors. Specific examples of viral vectors include, but are not limited to, vectors derived from pox viruses, retroviruses, SV40 virus, adenovirus, adeno-associated virus, HIV-1 virus, CMV, or herpes viruses. Once introduced into a host cell, the vector can remain episomal or may become chromosomal (i.e., incorporated into the genome of the host cell. A vector can include the radio-protective polypeptide encoding nucleic acid in a form suitable for expression of the nucleic acid in a host cell. Typically, the expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of polypeptide expression, and the like. In yet another refinement, the expression vector includes features that help prolonged expression of radio-protective peptides. Such features include, but are not limited to, changing codon usage from plant to mammal, ATG start codon preceded by a Kozak box (see for example U.S. Pat. No. 6,274,136), and attempted compliance with the Varshasysky N-end rule to slow cellular degradation (see for example, A. Varshaysky. The N-end rule pathway and regulation by proteolysis. Protein Science 2011 20:1298-1345; hereby incorporated by reference). In this regard, the polypeptide includes an N-terminal amino acid selected from the group consisting of valine, glycine, proline, isoleucine, threonine, and leucine.

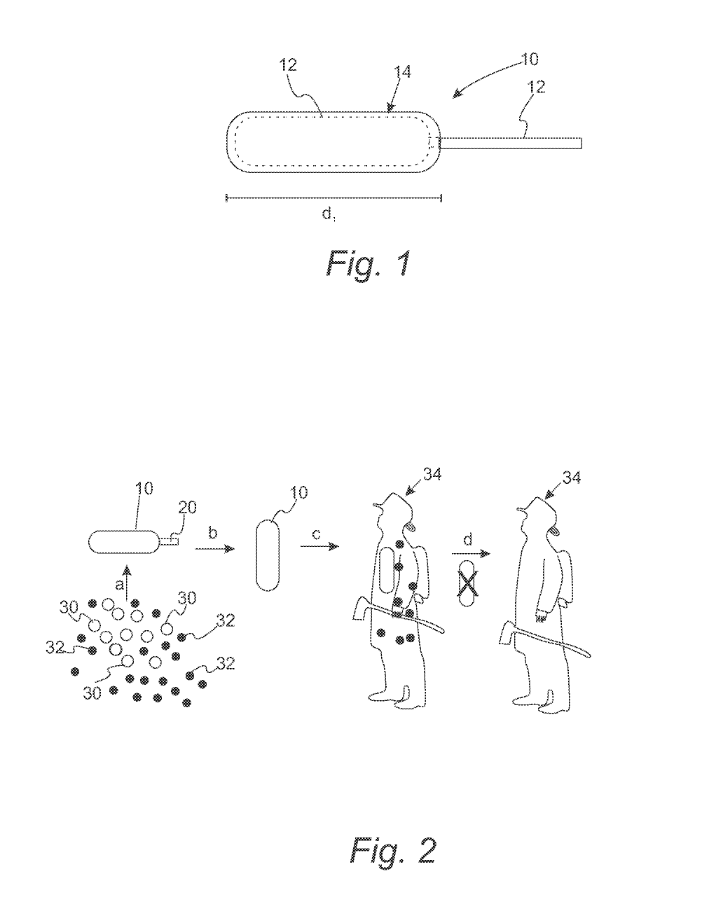

FIG. 1 provides a schematic illustration of an implantable device for delivering the transduced mammalian cells set forth above. A particularly useful example of such a device is the TheraCyte™ implantable device. Device 10 includes chamber 12 having a porous surface layer 14. Port 20 is used to introduce cells into the chamber and then sealed. Examples of useful implantable devices are set forth in U.S. Pat. Nos. 5,733,336; 5,882,354, 8,278,106; and 5,653,756; the entire disclosures of which are hereby incorporated by reference. Typically, device 10 has a length d₁ of about 1.5 to 2 inches.

FIG. 2 provides a schematic of radioprotection protocol. The device of FIG. 1 is administered before possible exposure to ionizing radiation. In step a), mammalian cells 30 secreting polypeptides 32 are introduced into device 10 via port 20. Port 20 is sealed in set b). In step c), device 10 is implanted into human subject 34 prior to exposure to radiation. In step d), the device continuously delivers protection for at least 3 months before being removed. After removal, a new device can then be implanted. The TheraCyte™ device has been used to implant allogeneic tissue in humans for 14 months where they were well tolerated with no signs of infection or inflammation. For example, normal fibroblasts are transduced with a minigene designed to encode and secrete a BBI peptide. The cells are sequestered in a device, in order to protect them from the immune response of the recipient and to allow straightforward removal after deployment. The TheraCyte™ implantable device is commercially available from TheraCyte, Inc. located in Laguna Hills, Calif.

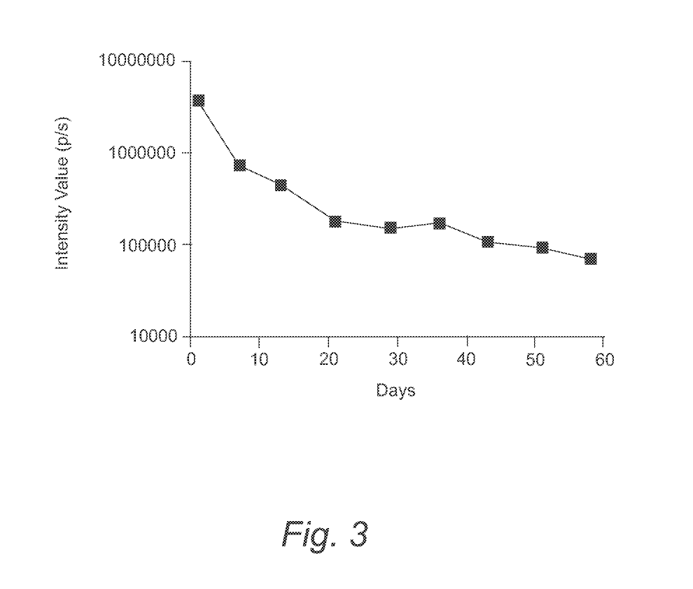

As set forth above, the methods of the invention utilize peptides which have established radio-protective properties (radio-protective peptides). Various embodiments of the invention utilizes several in vivo techniques as set forth in U.S. Pat. No. 6,274,136 which are used for treating multiple sclerosis. The entire disclosure of this patent is hereby incorporated by reference. For example, Anergix has developed technology which successfully delivers myelin peptides and treats Experimental Autoimmune Encephalomyelitis (EAE), a recognized model for multiple schlerosis. This technology allows for a secreted transgenic peptide to be detected by mass spectrometry and for expression vectors encoding signal sequences to increase peptide secretion. Transgene expression is detected in vivo for at least 8 weeks as shown in FIG. 3. FIG. 3 provides survival of transduced human fibroblasts contained within a TheraCyte device and implanted in mice. In this figure, 106 transduced human fibroblast cells (BJ line from ATCC) secreting luciferase were loaded into the TheraCyte™ device, implanted in mice and imaged once a week using an In vitro Image System (IVIS, Xenogen, Alameda, Calif.). For signal quantification, photons were obtained from the area of the implant.

The following examples illustrate the various embodiments of the present invention. Those skilled in the art will recognize many variations that are within the spirit of the present invention and scope of the claims.

Creation and Characterization of Transduced Murine Fibroblast Lines that Secrete Radioprotective Peptides.

Experiment 1: Normal (diploid) murine fibroblast line are transduced with a lentiviral vector carrying a minigene construct encoding a peptide from the Bowman Birk protease inhibitor (BBI) that has been demonstrated to be radioprotective. The BBI peptide itself has been shown to have radioprotective capability which makes it an ideal candidate to test our technology for in vivo continuous administration of a radioprotector. Since the radioprotective mechanism of action of this peptide is linked to repair of radiation-induced DNA damage, a nuclear localization signal (NLS) is added to assess whether presence of the targeting sequence increases the radioprotective effect.

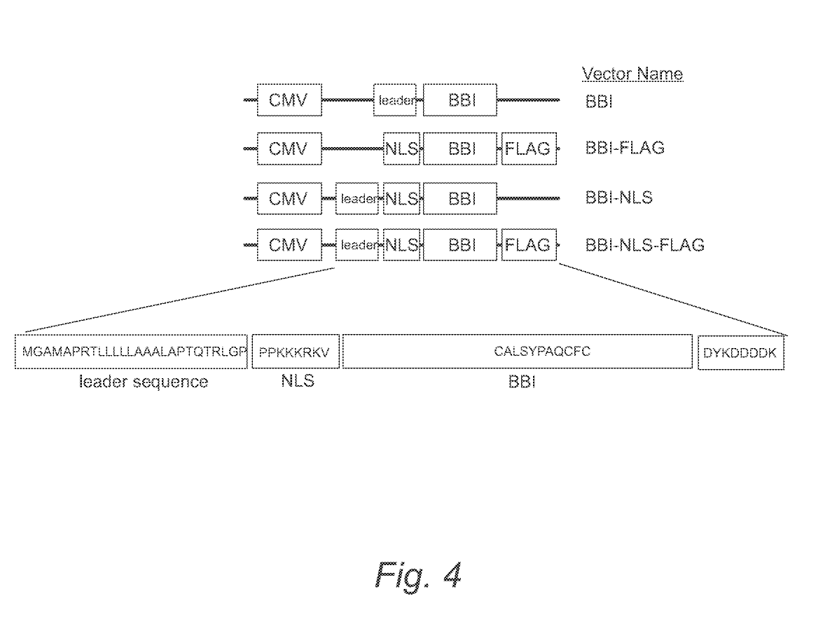

Minigene sequences encoding the BBI peptide are cloned into a HIV-1-derived lentiviral vector under control of the strong CMV promotor. This is an IRES-containing bicistronic vector that allows the simultaneous expression of our BBI minigene and the puromycin resistance gene from the same RNA transcript. The gene construct also encodes the FLAG tag sequence to enable detection and quantification of the peptide. Gene constructs without the FLAG sequence are also made to confirm that the FLAG sequence does not interfere with the radioprotective activity of the peptide. FIG. 4 provides diagrams of retroviral vectors. (NLS: nuclear localization sequence. The BBI sequence with radioprotective activity is shown in bold.). In this Figure, the leader sequences is provided by:

SEQ ID NO: 3
MGAMAPRTLLLLLAAALAPTQTRLGP
and the NLS by:
SEQ ID NO: 4
PPKKKRKV

In the present example, minigene DNA sequences are constructed from synthetic oligonucleotides and cloned into pLV67 lentiviral vector by GeneCopoeia (Rockville, MD). The mouse non-transformed fibroblast cell line, LBW, is transduced with the viral supernatant and cultured media containing puromycin for 2 weeks to select for stable transductants. Peptide secretion into the supernatant are quantified by ELISA assay using anti-FLAG antibodies. The ELISA assays provide a measure of the amount of peptide/number of transduced cells/time period. Custom synthesized BBI-FLAG peptide (NeoBioscience, Cambridge, Mass.) are used as a positive control and to prepare the standard curve to determine concentration.

Experiment 2: Determine the Radioprotective Capacity of the Peptide-Secreting Transduced Fibroblasts in an in vitro Cell Survival Model.

A transwell system is used to determine whether murine cell lines are protected from irradiation by co-culture with BBI peptide-secreting fibroblasts. BBI peptide has been shown to be radioprotective in vitro when administered to fibroblast cells prior to irradiation. In this experiment, the protective capability of the BBI-secreting fibroblasts is tested. This experiment assesses whether peptide-secreting fibroblasts exert a differential radioprotective effect by testing their ability to enhance survival of different cell types from lethal irradiation. Cell survival curves for each cell line are generated by a classical clonogenic assay. Radiation induced DNA damage is assessed by the comet assay and γ-H2AX assay. The plating efficiency for each cell line (transduced fibroblast (LBW-BBI), fibroblast (LBW1B2), liver epithelial (CCL9.1) (ATCC), kidney epithelial (TCMK-1)(ATCC), and bone marrow stroma (D1)(ATCC)) are determined by seeding cells at a low density and counting resulting colonies. For irradiation, target cells are seeded into 6-well tissue culture plates and for “with treatment” wells, transduced fibroblasts secreting the BBI peptide are placed in the transwell insert and the cells co-incubated for 24 hours, allowing time for target cell exposure to the secreted BBI peptide. In order to evaluate the effect of irradiation on transduced cells, these cells are also placed in a well and irradiated. Following incubation, the cells are irradiated (0-10Gy in 2 Gray steps). The exposures are performed using an XRAD 320ix x-ray machine set at 250 kvp, 16 mA, using a 10×10 cm field size and an SSD of 50 cm. The dose rate under these conditions is 2.51 Gray/minute. The exposures are controlled by a built-in computer system that uses an onboard parallel plate ionization chamber to monitor the dose as it is given. Immediately post-irradiation, the flasks of cells are returned to the laboratory (in a closed pre-warmed container to keep daylight from reaching the cells) where they are trypsinized (0.1% trypsin) and counted in preparation for being plated for the clonogenic survival assay. The following assays are then performed:

Clonogenic assay: Depending on the radiation dose given, cells are diluted in growth medium/10% fetal calf serum and seeded into three 10 cm diameter petri dishes such that 100 and 200 colonies/dish develop and are incubated at 37° C. for 10-14 days. Cells are then stained with crystal violet and the colonies with more than 50 cells are counted. To determine the surviving fraction, the average number of colonies in the three dishes are divided by the number of cells put in the dish and corrected for plating efficiency determined in a zero treated control. The Surviving fraction as a percent is plotted on a log scale against dose in Gray on a linear scale to give a dose response curve. Dose modifying factors (DMF) are determined from radiation survival curves by taking the ratio of radiation doses at a given survival level (BBI-treated plus radiation dose divided by the control radiation dose). DMF values >1 would indicate protection. The remaining cells not used in the clonogenic assay are split between two tubes and placed on ice until they can be used to assess DNA damage by the comet assay and the γH2AX assays.

Comet assay. This assay, also called single cell gel electrophoresis (SCGE), is used to quantify and analyze overall DNA damage in individual cells. In this study, a kit produced by Trevigen (Gaithersburg, Md.) that has standardized all the technical and chemical requirements to give consistent results shown by a fluorescent tail of DNA by each cell is used. The tail (comet) produced behind each cell is a result of fragmentation of the DNA by the radiation exposure. Once the tail is photographed and data collected for about a 100 cells per sample, the length of tail and the tail moment can be computed using appropriate software which allows a measure of the amount of DNA damage.

γH2AX assay. The second sample of cells put aside from each radiation dose are used to look for γH2AX foci. A member of the histone H2A family, H2AX, becomes extensively phosphorylated within minutes of DNA damage and forms foci at DSB sites. A γH2AX activation kit (Thermo Scientific, Rockford, Ill.) wherein γH2AX foci within a cell nucleus fluoresce green and are counted using a fluorescent microscope is used. Foci are enumerated in a minimum of 50 cells per sample; after subtracting any foci found in control (untreated cells or cells only treated with BBI but not radiation) the number of DSBs present can be determined. The effect of BBI on DSB formation is determined after increasing doses of radiation. This is then compared to the survival results.

Anticipated Results/Possible Pitfalls: BBI peptide added to cell culture prior to irradiation has been shown to protect fibroblasts from radiation-induced cell death in vitro as a result of an enhanced DNA repair. By co-culturing BBI peptide-secreting fibroblasts with target cells prior to irradiation enhanced cell survival is anticipated compared to target cells cultured alone. Although the transduced cells are also exposed to radiation, the production of BBI peptide prior to irradiation should protect the target cells. From these experiments, it is possible to determine whether targeting the BBI peptide to the nucleus helps protect not only the transduced cells from radiation damage but also enhances survival of the target cells. When added directly to the tissue culture medium, BBI peptide at a concentration of 20 μM protected fibroblasts from ionizing radiation. It is believed that less peptide is necessary in our delivery system; since peptide is continuously produced a high initial concentration is not necessary to avoid digestion by proteases present in the tissue culture supernatant. However, failure of the BBI peptide-secreting fibroblasts to provide radioprotection could be due to low concentration. To increase peptide, the co-incubation time is increased and/or a vector to carry multiple copies of the BBI peptide minigene is designed. The Comet assay and the γH2AX assays provide a semi-quantitative and quantitative analysis of total DNA damage and DSB damage present after radiation alone and radiation in the presence of BBI. The role of BBI in reducing DNA damage is determined and compared to how much reduction in cell killing occurred from a given dose of radiation and if the effects vary as the dose is increased. In vitro experiments are repeated three times with statistical analysis based on regression models being performed allowing comparisons over time and across experimental conditions.

Experiment 3: Determine the Radioprotective Capacity of the Peptide-Secreting Transduced Fibroblasts in an in vivo Model.

BBI peptide-secreting fibroblasts are sequestered in TheraCyte devices and implanted in mice that are then irradiated. In this experiment, transduced cells that showed protection in vitro are assessed for their ability to provide protection in vivo. BBI peptide-secreting fibroblasts are loaded into TheraCyte devices (1×10⁶ cells/device). Devices are held in tissue culture medium (RPMI/10% FCS) until implanted (up to 36 hours). 8-12 week old B6 female mice are anesthetized by inhalation of 1.5% isoflurane-98.5% O₂ (Abbott Laboratories, North Chicago, Ill.), and the implant placed subcutaneously on the back. Seven days after implantation mice are irradiated with the indicated total body doses. An XRAD 320ix irradiator (Precision X-Ray, Stanford, Conn.) is used as the ionizing radiation source. Following irradiation, mice are monitored for 4 weeks. Weight is recorded every 2-3 days. A 20% weight loss is used as a signal to euthanize the mouse and is recorded as death due to radiation exposure. Blood is drawn every 5-7 days to assess DNA damage (Comet assay and γH2AX assay—see Experiment 2) and white blood cell and platelet count. After 4 weeks, mice are euthanized. Mice that received cells producing BBI-FLAG peptide have spleen, lymph nodes, liver and kidneys removed for immunohistochemistry using anti-FLAG antibodies. Primary endpoints for this experiment are mouse weight, the results of the Comet and γH2AX assays, white blood cell and platelet counts, and survival. Weight, assay results and cell counts (possibly after transformation to permit parametric statistical analysis) are plotted over time—for each mouse individually and then for the aggregate of each of the 20 experimental conditions. Regression models that accommodate repeated measures are used to compare the 5 treatments groups as a function of radiation dose and time; contrasts are used to test for specific differences. Survival is summarized using Kaplan-Meier plots and the Cox proportional hazards model (if the assumption of proportional hazards is appropriate). Adjustments are made for multiple comparisons; pair-wise comparisons are undertaken only if the overall main effect is significant. All experiments are done twice with N=5 for each group (i.e. 10 per group). To evaluate the statistical power with 10 mice, comparison of all 5 treatments at one radiation dose and at one time point (the overall analysis has more power) is considered; with a 0.05-level F-test based on a one-way analysis of variance with 5 groups, there is at least 82% power when the most effective cell line improves the outcome over the control by 1.67σ, where σ, the standard deviation, represents the intrinsic mouse-to-mouse variation; for a vector to be effective, large effects that are at least of this magnitude are required. Enhanced survival in mice receiving BBI-secreting fibroblasts is anticipated. The primary mechanism for increasing overall peptide production is to create a minigene construct that encodes multiple BBI peptide repeats.

| | # Animals per radiation dose* | | | | |
|---|---|---|---|---|---|
| Cell Line | 2 Gy | 4 Gy | 6 Gy | 8 Gy | |
| No treatment | 5 | 5 | 5 | 5 | Weight is recorded |
| LBW-empty | 10 | 10 | 10 | 10 | every 2-3 days for |
| vector control | | | | | 4 weeks |
| LBW-BBI-FLAG | 10 | 10 | 10 | 10 | Blood is sampled |
| LBW-BBI | 10 | 10 | 10 | 10 | every 5-7 days for |
| LBW-NLS-BBI | 10 | 10 | 10 | 10 | DNA damage |
| | | | | | assessment and |
| | | | | | white blood cells |
| | | | | | counted |

*In vivo experiments are repeated two times each time with 5 mice per group

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method of reducing a symptom of radiation exposure in a subject, the method comprising:
introducing mammalian cells into the subject, the mammalian cells transduced with an expression vector including a polynucleotide encoding polypeptide that is protective against radiation and an expression control sequence operably linked to the polypeptide, the mammalian cells expressing the polypeptide, at least 10% of the polypeptide's amino acid residues being selected from the group consisting of cysteine, tryptophan, phenylalanine, tyrosine and combinations thereof.

2. The method of claim 1 wherein the polypeptide includes from about 10 to about 40 amino acid residues.

3. The method of claim 1 wherein the polypeptide includes an N-terminal amino acid selected from the group consisting of valine, glycine, proline, isoleucine, threonine, and leucine.

4. The method of claim 1 wherein a minigene construct is inserted into the mammalian cells, the minigene construct including the polypeptide and the expression control sequence.

5. The method of claim 4 wherein the expression control sequence includes a signal sequence.

6. The method of claim 5 wherein the signal sequence targets caveolae, endosomes, nuclear membranes, cell membranes, other cell vesicle membranes, and combinations thereof.

7. The method of claim 5 wherein the expression control sequence includes an ATG start codon preceded by a Kozak box.

8. The method of claim 5 wherein the minigene construct includes a FLAG tag sequence

9. The method of claim 1 wherein the polypeptide includes the polypeptide having SEQ. ID. NO.: 1 (Bowman Birk protease inhibitor) or 10 to 40 amino acid residues from the polypeptide having SEQ. ID. NO.: 1 (Bowman Birk protease inhibitor).

10. The method of claim 1 wherein the cells are sequestered in a chamber that is removed at the end of a deployment.

11. The method of claim 1 wherein the mammalian cells are selected from the group consisting of fibroblasts, autologous B cells, stem cells, and combinations thereof.

12. The method of claim 1 wherein a plurality of mammalian cells having different polynucleotides encoding polypeptide that are protective against radiation are introduced into the subject.

13. A device for delivering radiation protecting polypeptides to a subject comprises:
a chamber; and
mammalian cells sequestered in the chamber, the mammalian cells transduced with an expression vector including a polynucleotide encoding a polypeptide that is protective against radiation and an expression control sequence operably linked to the polypeptide, the mammalian cells expressing the polypeptide, at least 10% of the polypeptide's amino acid residues being selected from the group consisting of cysteine, tryptophan, phenylalanine, tyrosine and combinations thereof.

14. The device of claim 13 wherein the polypeptide includes from about 10 to about 40 amino acid residues.

15. The device of claim 13 wherein the polypeptide includes an N-terminal amino acid selected from the group consisting of valine, glycine, proline, isoleucine, threonine, and leucine.

16. The device of claim 13 wherein a minigene construct is inserted into the mammalian cells, the minigene construct including the polypeptide and the expression control sequence.

17. The device of claim 16 wherein the expression control sequence includes a signal sequence targeting caveolae, endocytosis, nuclear membranes, cell membranes, other cell vesicle membranes, or combinations thereof.

18. The device of claim 13 wherein the polypeptide includes the polypeptide having SEQ. ID. NO.: 1 (Bowman Birk protease inhibitor) or 10 to 40 amino acid residues from the polypeptide having SEQ. ID. NO.: 1 (Bowman Birk protease inhibitor).

19. The device of claim 13 wherein the mammalian cells are selected from the group consisting of fibroblasts, autologous B cells, stem cells, and the like.

20. The device of claim 13 wherein a plurality of mammalian cells having different polynucleotides encoding polypeptide that are protective against radiation are sequestered in the chamber.

21. A cultured cell comprising:
a polynucleotide encoding polypeptide that is protective against radiation, at least 10% of the polypeptide's amino acid residues are selected from the group consisting of cysteine, tryptophan, phenylalanine, tyrosine and combinations thereof; and
an expression control sequence operably linked to the polynucleotide, the cultured cell expressing the polypeptide.

22. The cultured cell of claim 21 wherein the polypeptide includes from about 10 to about 40 amino acid residues.

23. The cultured cell of claim 21 wherein the expression control sequence includes a signal sequence targeting caveolae, endosomes, nuclear membranes, cell membranes, other cell vesicle membranes, and combinations thereof.

24. The cultured cell of claim 23 wherein the expression control sequence includes an ATG start codon preceded by a Kozak box.

25. The cultured cell of claim 21 wherein the polypeptide includes the polypeptide having SEQ. ID. NO.: 1 (Bowman Birk protease inhibitor) or 10 to 40 amino acid residues from the polypeptide having SEQ. ID. NO.: 1 (Bowman Birk protease inhibitor).


SEQUENCE LISTING (5 sequences)

SEQ ID NO: 1 - protein, 71 aa, Soybean
DDESSKPCCDQCACTKSNPPQCRCSDMRLNSCHSACKSCICALSYPAQCFCVDITDFCYEPCKPSEDDKEN

SEQ ID NO: 2 - protein, 11 aa, Artificial Sequence
  portion of Soybean Bowman Birk protease inhibitor
CALSYPAQCFC

SEQ ID NO: 3 - protein, 26 aa, Artificial Sequence
  leader or signal sequence
MGAMAPRTLLLLLAAALAPTQTRLGP

SEQ ID NO: 4 - protein, 8 aa, Artificial Sequence
  nuclear localization sequence
PPKKKRKV

SEQ ID NO: 5 - protein, 8 aa, Artificial Sequence
  FLAG sequence
DYKDDDDK